(19) United States
(12) Patent Application Publication
(10) Pub. No.: US 2008/0027401 A1
(43) Pub. Date: Jan. 31, 2008

(54) VASCULAR ACCESS DEVICE FILTRATION

(75) Inventors: David Ou-Yang, Woodbury, MN (US); S. Ray Isaacson, Roy, UT (US); Marty L. Stout, South Weber, UT (US); Glade H. Howell, Sandy, UT (US)

Correspondence Address: David W. Highet;Becton, Dickinson and Company, (Metcalf Intellectual Property Law, LLC), 1 Becton Drive, MC 110, Franklin Lakes, NJ, 07417-1880, US

(73) Assignee: BECTON, DICKINSON AND COMPANY, Franklin Lakes, NJ (US)

(21) Appl. No.: 11/829,009

(22) Filed: Jul. 26, 2007

Related U.S. Application Data
(60) Provisional application No. 60/820,703, filed on Jul. 28, 2006.

(51) Int. Cl. A61M 25/00

(52) U.S. Cl. 604/265

(57) ABSTRACT

A vascular access device includes an interior chamber for receiving a fluid and a filter within the interior chamber for filtering a pathogen within the fluid. A method of filtering a pathogen in a vascular access device includes providing a vascular access device having an interior chamber for receiving a fluid, providing a filter within the interior chamber of the vascular access device to move the fluid through the filter, and filtering a pathogen as the fluid moves through the filter.

This application claims the benefit of U.S. Provisional Application No. 60/820,703, filed Jul. 28, 2006, entitled VASCULAR ACCESS DEVICE FILTRATION, which is incorporated herein by reference.

### BACKGROUND OF THE INVENTION

The present disclosure relates to infusion therapy with antimicrobial vascular access devices. Infusion therapy is one of the most common health care procedures. Hospitalized, home care, and other patients receive fluids, pharmaceuticals and blood products via a vascular access device inserted into the vascular system. Infusion therapy may be used to treat an infection, provide anesthesia or analgesia, provide nutritional support, treat cancerous growths, maintain blood pressure and heart rhythm, or many other clinically significant uses.

Infusion therapy is facilitated by a vascular access device. The vascular access device may access a patient's peripheral or central vasculature. The vascular access device may be indwelling for short term (days), moderate term (weeks), or long term (months to years). The vascular access device may be used for continuous infusion therapy or for intermittent therapy.

A common vascular access device is a plastic catheter that is inserted into a patient's vein. The catheter length may vary from a few centimeters for peripheral access to many centimeters for central access. The catheter may be inserted transcutaneously or may be surgically implanted beneath the patient's skin. The catheter, or any other vascular access device attached thereto, may have a single lumen or multiple lumens for infusion of many fluids simultaneously.

The proximal end of the vascular access device commonly includes a Luer adapter to which other medical devices may be attached. For example, an administration set may be attached to a vascular access device at one end and an intravenous (TV) bag at the other. The administration set is a fluid conduit for the continuous infusion of fluids and pharmaceuticals. Commonly, an IV access device is a vascular access device that may be attached to another vascular access device, closes or seals the vascular access device, and allows for intermittent infusion or injection of fluids and pharmaceuticals. An IV access device may include a housing and a septum for closing the system. The septum may be opened with a blunt cannula or a male Luer of a medical device.

Complications associated with infusion therapy may cause significant morbidity and even mortality. One significant complication is catheter related blood stream infection (CRBSI). An estimate of 250,000-400,000 cases of central venous catheter (CVC) associated BSIs occur annually in US hospitals. Attributable mortality is an estimated 12%-25% for each infection and a cost to the health care system of $25,000-$56,000 per episode.

Vascular access device infection resulting in CRBSIs may be caused by failure to regularly clean the device, a non-sterile insertion technique, or by pathogens entering the fluid flow path through either end of the path subsequent to catheter insertion. Studies have shown the risk of CRBSI increases with catheter indwelling periods. When a vascular access device is contaminated, pathogens adhere to the vascular access device, colonize, and form a biofilm. The biofilm is resistant to most biocidal agents and provides a replenishing source for pathogens to enter a patient's bloodstream and cause a BSI. Thus, what are needed are systems, devices, and methods to reduce the risk and occurrence of CRBSIs.

### BRIEF SUMMARY OF THE INVENTION

The present invention has been developed in response to problems and needs in the art that have not yet been fully resolved by currently available vascular access systems, devices, and methods. Thus, these systems, devices, and methods are developed to reduce the risk and occurrence of CRBSIs.

A medical device may be a vascular access device that includes an interior chamber for receiving a fluid and a filter within the interior chamber for filtering a pathogen within the fluid. The medical device may also include an antimicrobial agent within the interior chamber. The filter may be impervious to the antimicrobial agent and border the antimicrobial agent on at least a first side. The filter may include an electrical multilayer screen, a biocide barb, and/or multiple layers of biocide barbs. The filter may prevent the passage of any agent the size of a pathogen. The filter may be a silver-coated wire mesh.

A method of filtering a pathogen in a vascular access device includes providing an interior chamber in the vascular access device for receiving a fluid, providing a filter within the interior chamber of the vascular access device, moving the fluid through the filter, and filtering a pathogen as the fluid moves through the filter. The method may also include providing an antimicrobial agent within the interior chamber and bordering the antimicrobial agent with the filter on at least a first side of the filter to make it impervious to the antimicrobial agent.

The method of filtering may include electrocuting the pathogen as it moves through the filter, cutting the pathogen as it moves through the filter, and/or preventing the passage through the filter of any agent the size of the pathogen. The filter may include multiple layers of biocidal barbs. The filter may include a silver-coated wire mesh.

A medical device may include means for accessing the vascular system of a patient and means for filtering a pathogen. The means for filtering the pathogen is located within the means for accessing the vascular system of the patient. The means for filtering a pathogen may include a means for killing a pathogen bordered by a means for retaining the means for killing within the means for accessing. The means for filtering may include means for electrocuting the pathogen, means for cutting the pathogen, means for preventing the passage of any agent the size of the pathogen, and/or a biocidal coating.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to limit the scope of the invention,

FIG. 1 is a perspective view of an extravascular system connected to the vascular system of a patient.

FIG. 2 is a cross section view of a vascular access device having an antimicrobial agent bounded or bordered by two filters.

FIG. 3 is a cross section view of a vascular access device having an antimicrobial agent bounded by a filter.

FIG. 4 is a cross section view of an electrical multi-layered screen.

FIG. 5 is a cross section view of a septum with biocidal barbs.

FIG. 6 is a close-up partial cross section view of a portion of the septum of FIG. 5.

FIG. 7A is a partial cross section view of the tip of a separate access device, a close-up view of a biocidal layer, a further close-up view of the barbs of the layer, and a further close-up view of a barb and a pathogen.

FIG. 7B is a cross section view of taken along line 7A-7A of FIG. 7A.

FIG. 7C is a close up view illustrating biocide barbs located on a biocide grid.

FIG. 7D is a further close up view of an individual biocide barb.

FIG. 8 is a side view of a vascular access device and a filter.

FIG. 9 is a transparent side view of a vascular access device and a silver-coated wire mesh.

### DETAILED DESCRIPTION OF THE INVENTION

The presently preferred embodiments of the present invention will be best understood by reference to the drawings, wherein like reference numbers indicate identical or functionally similar elements. It will be readily understood that the components of the present invention, as generally described and illustrated in the figures herein, could be arranged and designed in a wide variety of different configurations. Thus, the following more detailed description, as represented in the figures, is not intended to limit the scope of the invention as claimed, but is merely representative of presently preferred embodiments of the invention.

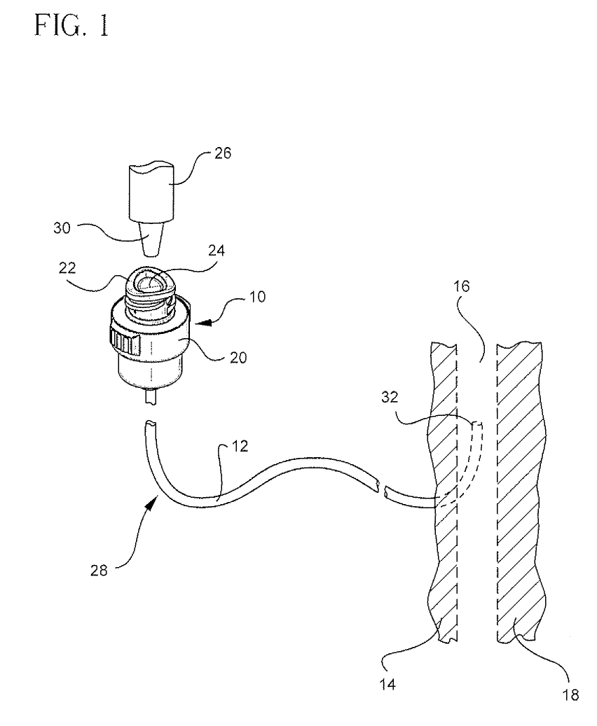

Referring now to FIG. 1, a vascular access device (also referred to as an extravascular device, intravenous access device, access port, and/or any device attached to or functioning with an extravascular system) 10 is used to introduce a substance via a catheter 12 across the skin 14 and into a blood vessel 16 of a patient 18. The vascular access device 10 includes a body 20 with a lumen and a septum 22 placed within the lumen. The septum 22 has a slit 24 through which a separate extravascular device 26, such as a syringe, may introduce a substance into the vascular access device 10.

The device 10 also includes a filter (discussed with reference to the figures below) capable of filtering a pathogen within the vascular access device 10, including the catheter 12 and the end 32 of the catheter 12, and/or the extravascular system 28 to which the vascular access device 10 is connected. The filter filters the pathogen to decrease the incidence of blood stream infections in patients to whom the vascular access device 10 or any other device on an extravascular system 28 is attached.

A pathogen may enter the device 10 or system 28 in any of a number of ways. For example, a pathogen may reside within the device 10 or system 28 prior to first use. A pathogen may also be introduced into the device 10 from the external surface of the device, the external surface of a separate device 26, and/or the surrounding environment when a structure such as a tip 30 of the separate device 26 is inserted into the device 10 through the slit 24 of the septum 22. A pathogen may be introduced within fluid that is infused into the system from a separate device 26. Finally, a pathogen may be introduced from a blood vessel 16 into the system 28 by entering through the end 32 of the catheter 12 during a blood draw or a period of blood reflux when the device 10 is in use. Filters may thus be placed along any portion of the fluid path along the interior of the system 28 in order to control pathogenic flow along the fluid path, as desired.

As described throughout this specification, the filter controls pathogenic flow by exerting any combination of the following actions upon a pathogen: trapping, securing, electrocuting, electrifying, killing, attracting to a location, repelling from a location, degrading, frustrating, shearing, cutting, fragmenting, preventing growth or proliferation, radiating, and/or any other similar process or action. Further, pathogens include any agent that causes a disease or otherwise harms or has the potential to harm a patient if received into the vascular system of that patient, including a pathogen, bacterium, parasite, microbe, biofilm, fungus, virus, protein feeding a pathogen, protozoan, and/or other harmful microorganisms and/or agents and products thereof.

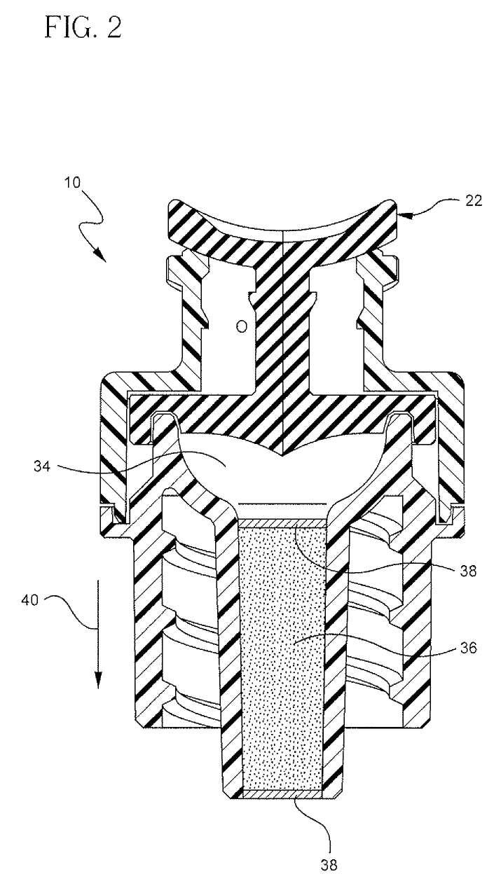

Referring now to FIG. 2, a vascular access device 10 includes an interior chamber 34. A high concentration antimicrobial agent 36 is bounded by two filters 38 within the interior chamber 34. The filters 38 are impervious to the antimicrobial agent 36 such that the antimicrobial agent cannot escape the boundaries of the filters 38. However, the filters 38 permit fluid and pathogens to travel through them across the first filter 38 into the antimicrobial agent 36 and ultimately across the second filter 38 while traveling in a direction 40. When a pathogen enters into the bounded chamber where the antimicrobial agent 36 is present, the antimicrobial agent 36 kills or otherwise harms the pathogen. The pathogen may then either continue to reside within the bounded chamber or may pass through the second filter 38 and ultimately into a patient in a harmless state.

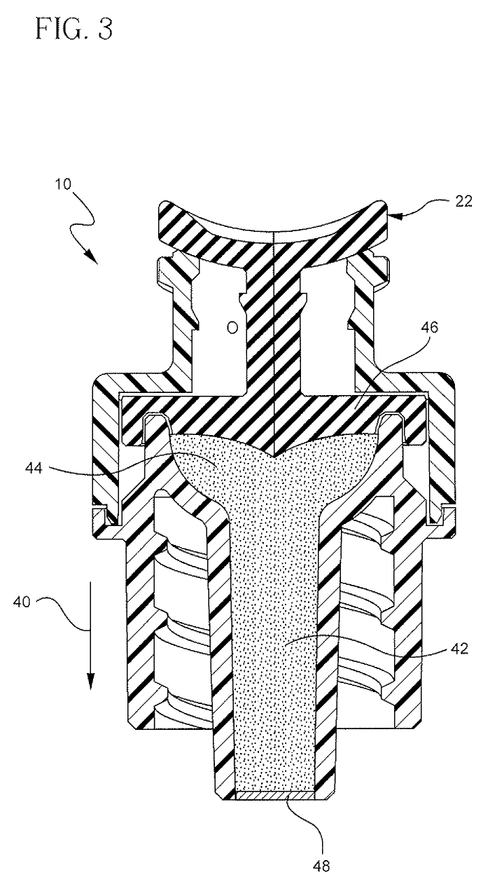

Referring now to FIG. 3, a vascular access device 10 may include an antimicrobial agent 42 in high concentration within its interior chamber 44. The antimicrobial agent 42 is bounded on an upper end by the floor 46 of the septum 22 of the device 10. The antimicrobial agent 42 is bounded on its lower end by a filter 48 that is impervious to the antimicrobial agent 42, but permits passage of other fluids and pathogens across its membrane. Similar to the embodiment described with reference to FIG. 2, the present embodiment permits a fluid containing a pathogen to travel into the environment of the antimicrobial agent 42 where the pathogen is killed or otherwise harmed.

A number of embodiments that are alternative to those described with reference to FIGS. 2 and 3 are included within the scope of the present invention. For example, the filters 38 and 48 can be formed of any filtration material capable of performing the required function of the filters 38 and 48. For example, the filter material may be a metal or a plastic screen, a porous material or non-woven material, or a filter paper such as a synthetic filter paper. Various filter materials may be used to adjust the flow rate of a fluid across the filter. The concentration of the antimicrobial agents 36 and 42 may be up to 100 percent. Such antimicrobial agents can be blended into a polymeric material that is hydrophilic or hydrophobic and includes good diffusivity. The antimicrobial agents may also be encapsulated inside of an organic, inorganic, or polymeric shell, which has a controllable diffusion rate for the agent. Such diffusion may occur at various rates depending on the rate of infusion of a fluid into the device 10 and/or the type of fluid infused into the device 10.

The antimicrobial agents may also be coated onto the surface of a number of micro-porous particles or beads. The antimicrobial agents may also be placed on or coated onto a filter that is bounded by the filters 38 and 48 or any filter-like material including a film, fiber, a metal or plastic screen, a porous or non-woven material, and/or a paper, including a synthetic paper. The antimicrobial materials may also be impregnated or salivated into any of the above materials.

The antimicrobial agents 36 and 42 and other antimicrobial agents discussed throughout this specification, may include any of the following antimicrobial agents alone or in combination, as shown in Table 1 below.

TABLE 1

| | Antimicrobial Mechanism of | |
|---|---|---|
| Technology/Company | Action | Active Ingredient |
| Alexidine | Bisbiguanide/Antiseptic | Alexidine |
| AMERICAL | Halogen/Antiseptic | Iodine |
| (Merodine) | | |
| Angiotech | Antimicrobial/ | 5-Flurouracil |
| Pharmaceuticals | Antineoplastic | |
| Apacidar (SGA) | Metals & Salts | Silver |
| Arglaes (Giltech) | Metals & Salts | Silver |
| Arrow Howes CHG | Bisbiguanide/Antiseptic + antibiotic | Chlorhexidine and Silver |
| and AgSD | | Sulfadiazine |
| Bactifree | Metal & Salts | Silver |
| Bacterin | Metal | Silver Hydrogel |
| BASF PVP-I Dusted | Halogen/Antiseptic | Iodine |
| Gloves BD | | |
| Baxter American | Antiseptic and | Benzalkonium Chloride |
| Edwards | Anticoagulant | complexed Heparin |
| Benzalkonium | Quaternary | Benzalkonium Chloride |
| Chloride | Ammonium/Antiseptic | |
| Benzethonium | Quaternary | Benzethonium Chloride |
| Chloride | Ammonium/Antiseptic | |
| Bioshield (CATO | Halogen/Antiseptic | Iodine |
| Research) | | |
| BisBAL | Metal, mercury | Bismuth and 2,3 |
| | | dimercaptopropanol |
| | | a.k.a.dimercaprol, or British |
| | | anti-lewisite |
| CATO Research | Halogen/Antiseptic | Iodine |
| (Bioshield) | | |
| Chlorhexidine (and its | Bisbiguanide/Antiseptic | Chlorhexidine |
| salts) | | |
| Ciprofloxacin | Antibiotic | Ciprofloxacin |
| TDMAC Complex | | |
| BD | | |
| Cooke | TDMAC bound Antibiotic | Any antibiotic |
| Cosmocil | Bisbiguanide/Antiseptic | Cosmocil |
| Cyclodextrin | Nonstick surface | Cyclodextrin |
| Daltex | Bisbiguanide/Antiseptic | Chlorhexidine and Silver |
| | | Sulfadiazine |
| Dicloxacillin | Antibiotic | Dicloxacillin |
| TDMAC Complex | | |
| BD | | |
| EDTA, EGTA | Calcium Chelator | EDTA, EGTA |
| Epiguard (Iodine) | Halogen/Antiseptic | Iodine |
| Epitope (Iodine) | Halogen/Antiseptic | Iodine |
| ExOxEmis | Oxidative enzymes | Myeloperoxidase and |
| | | Eosinophil Peroxidase |
| Fusidic Acid | Antibiotic | Fusidic Acid |
| TDMAC Complex | | |
| BD | | |
| Gamma A | Specific Antibodies | Specific Antibodies |
| Technologies | | |
| Giltech | Metal & Salts | Silver |
| Glyzine | Metals & Salts | Zinc |
| Gold | Metal & Salts | Gold |
| Healthshield | Metal & Salts | |
| Heparin- | Antimicrobial/ | |
| Benzalkonium | Antithrombogenic | |
| Chloride | | |
| Hexyl Bromide | Metals & Salts | Hexyl Bromide |
| Implemed (Ag/Pt) | Metal & Salts | Silver/Platinum |
| Intelligent Biocides | Metals & Salts | Silver |
| Iodine | Halogen/Antiseptic | Iodine |
| Iodine Tincture | Halogen/Antiseptic | Iodine |
| Irgasan | Phenolic/Antiseptic | Triclosan |
| Johnson-Matthey | Metal | Silver |
| Kinetic Concepts | Metals & Salts | Silver |
| Luther Medical | Antibiotic | Polymyxin B |
| Lysozyme | Enzymatic Antibiotic | |
| Mediflex | Bisbiguanide/Antiseptic | Chlorhexidine/Isopropanol |
| Chlorhexidine | | |
| Gluconate Tincture | | |
| Merodine | Halogen/Antiseptic | Iodine |
| Microban | Antiseptic Polymer | Triclosan |
| Microbia | Antibiotic | “Natural” polypeptides |
| MicroFre | Metal & Salts | |
| Minocycline | Antibiotic | Minocycline Rifampin |
| Rifampin | | |
| Minocycline-EDTA | Antibiotic | Minocycline EDTA |
| Morton Bloom | Cidal Lipids | Free fatty acids |
| Novacal | Neutrophil Cidal Factors | Oxidative Enzymes |
| Octenidine | Bisbiguanide/Antiseptic | Octenidine |
| Oligon (Implemed | Metal & Salts | Silver/Platinum |
| Ag/Pt) | | |
| Olin Chemicals | Metal & Salts | Zinc |
| Omacide | Metal & Salts | Zinc |
| Omni Medical | Heterologous Antibodies | Antibodies |
| Orthophenyl phenol | Phenolic/Antiseptic | Orthophenyl phenol |
| (Lysol) | | |
| Phosphorus | Antimicrobial Polymer | Phosphorus |
| Polymyxin B (Luther) | Antibiotic | |
| PVP-I (Iodine) | Halogen/Antiseptic | Iodine |
| Quorem Sciences | Cell-signalling | Peptides |
| Rifampin | Antibiotic | Rifampin |
| Sangi Group America | Metal & Salts | Silver |
| SGA | Metal & Salts | Silver |
| Silver Chloride | Metal & Salts | Silver |
| Silver Nitrate | Metal & Salts | Silver |
| Silver Oxide | Metal & Salts | Silver |
| Silver Palladium | Metal & Salts | Silver |
| Spi-Argent | Metal & Salts | Silver |
| Spire | Metal & Salts | Silver |
| Surfacine | Metal & Salts | Silver |
| TCC (Triclocarban) | Phenolic/Antiseptic | Triclocarban |
| TCS (Triclosan) | Phenolic/Antiseptic | Triclosan |
| TDMAC | Antibiotics | Cephazolin, Cipro., |
| | | Clindamycin, Dicloxacillin, |
| | | Fusidic Acid, Oxacillin, |
| | | Rifampin |
| Triclocarban | Phenolic/Antiseptic | Triclocarban |
| Triclosan | Phenolic/Antiseptic | Triclosan |
| Vancomycin | Antibiotic | Vancomycin |
| Vancomycin-Heparin | Antibiotic | Vancomycin-Heparin |
| Vibax | Phenolic/Antiseptic | Triclosan |
| Vitaphore CHG | Bisbiguanide/Antiseptic | Chlorhexidine |
| coating | | |
| Vitaphore Silver Cuff | Metal & Salts | Silver |
| Zinc | Metal & Salts | Zinc |
| Zinc Omadine | Metal & Salts | Zinc |

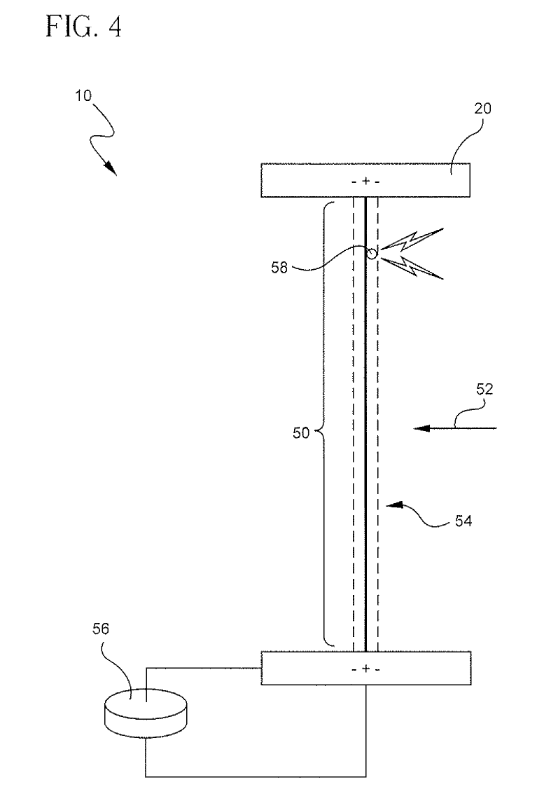

Referring now to FIG. 4, a vascular access device 10 includes a filter that is an electrical multi-layer screen 50 traversing a fluid path 52 within an interior chamber 54 inside the body 20 of the device 10. The electrical screen 50 includes multiple layers that are either positively or negatively charged by means of power supplied by a battery 56 connected in series with the screen 50. When a pathogen 58 such as a bacteria travels along the fluid path 52 and attempts to penetrate the screen 50, the size of the pathogen will cause it to come into contact or close proximity with layers of opposite charge in the screen 50. When the pathogen 58 is thus situated, it will complete a circuit between the two layers causing electricity to transfer from one layer across the pathogen into the other layer, electrocuting or otherwise electrifying the pathogen 58. When the pathogen 58 is electrocuted or electrified, the pathogen 58 is either killed or harmed to the point that it is rendered harmless to the vascular system of a patient.

The electrical screen 50 may be continuously powered by the battery 56 or other power source, and may be turned off by a device 10 operator during drug delivery along the fluid path 52 or during a blood draw along the same fluid path 52. When the screen 50 is not turned off, it delivers a continuous, small charge across the various layers of the screen 50 needed to kill or harm organisms as they attempt to penetrate the screen 50. The electrical multi-layered screen 50 may traverse a fluid path 52 or may reside in, on, around, or near any interior chamber 54 of the device 10.

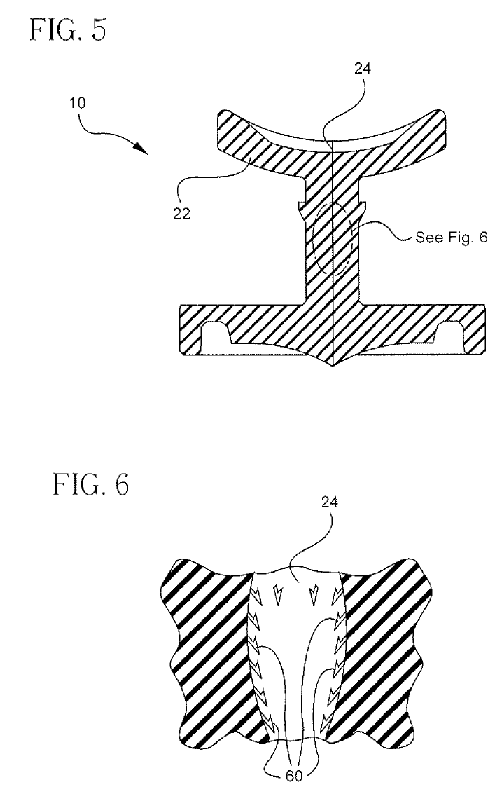

Referring now to FIG. 5, a vascular access device 10 includes a septum 22 with biocide barbs residing within a slit 24 of the septum 22.

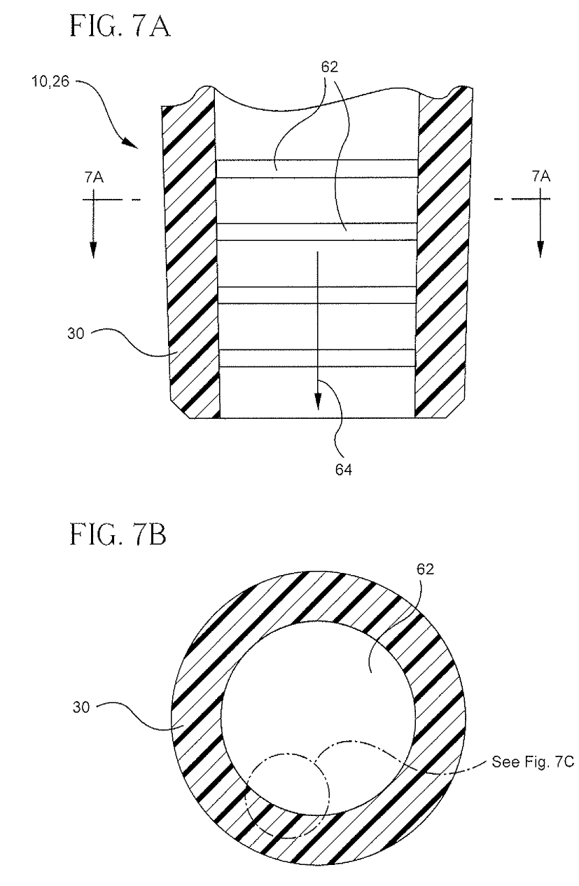

Referring now to FIG. 6, a close up view of the slit 24 of FIG. 5 is shown with the slit 24 shown in open position. The slit 24 includes biocide barbs 60 in a substantially enlarged view. The biocide barbs 60 will be small enough to be able to penetrate the cell of a pathogen and may include carbon nano-tubes. A sharp edge of each of the biocide barbs is sufficiently sharp and small to cut, pierce, shear, or otherwise fragment a surface, cell, or capsule of a pathogen as the pathogen travels at the normal speed of a fluid flow and comes into contact with a barb 60. When a cell wall of the pathogen is cut by a barb 60, the cell will become disarmed and/or ultimately die.

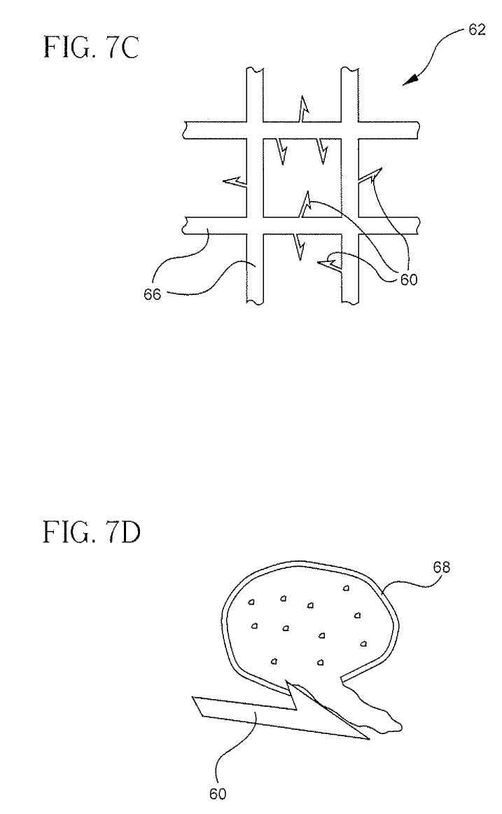

Referring now to FIG. 7A, a vascular access device 10 such as a separate access device 26 may include multiple layers of biocide barbs 62 along its fluid path 64. The layers 62 may be in the form of grids, sheets, materials, and/or other organized or asymmetrical groupings of biocide barbs similar to the biocide barbs 60 described with reference to FIG. 6. As fluid travels along the length of the tip 30 of the separate access device 26, any pathogen traveling along the fluid path 64 will be cut and will subsequently die as a result of its contact with a barb within one of the biocide layers 62.

Also shown in FIG. 7B is a close-up cross section view taken along lines A-A of the tip 30 revealing a biocide grid as one of the multiple layers 62. In FIG. 7C a further close-up view reveals the individual biocide barbs 60 located between the separate portions 66 of the biocide grid 62. In FIG. 7D an even further close-up view of an individual biocide barb 60 reveals the proportionate size of the barb in relation to a pathogen 68. The barb is shown penetrating or otherwise tearing the cell wall of the pathogen 68 in a manner which causes the internal contents of the pathogen 68 to exit its cell wall, initiating cell death of the pathogen 68.

The embodiments described with reference to FIGS. 5 through 7D will preferably be employed in a vascular access device that is used for fluid, but not blood, infusion into the vascular system of a patient. The barbs 60 of these embodiments should not be employed in combination with a blood draw or blood transfusion, as the barbs 60 may cause damage to the healthy blood cells within the fluid that is being transmitted.

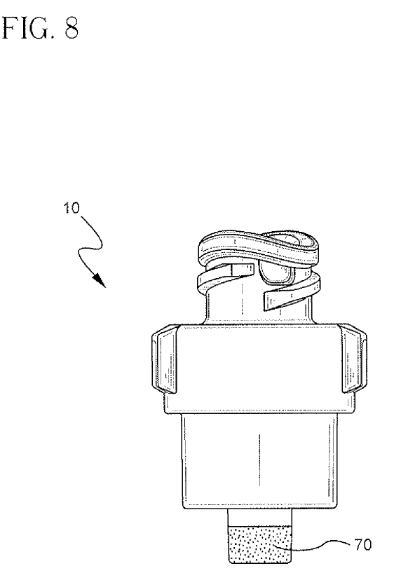

Referring now to FIG. 8, a vascular access device 10 includes a filter 70. The filter 70 is small enough to screen out any agent the size of a pathogen or other microbe. Such agents may be as small as, and larger than, any pathogen. The filter 70 may be included along any portion of the fluid path of the device 10 or any device that is connected in series with the device 10 along an extravascular system 28 (see FIG. 1).

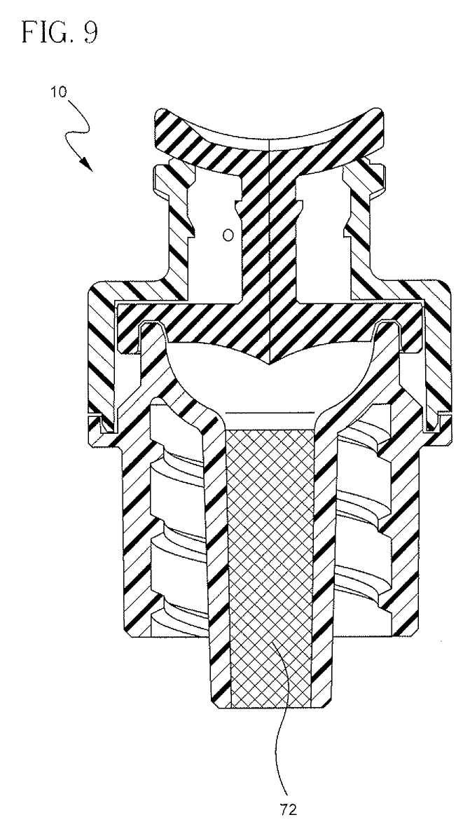

Referring now to FIG. 9, a vascular access device 10 includes a silver coated wire mesh as a filter 72. Since silver is a natural biocide for pathogens, as the pathogens pass through the silver coated wire mesh 72, they will be harmed or killed prior to infusion into the vascular system of a patient. Any other material that is coated with any of the agents mentioned in Table 1 or any other biocidal agent with similar properties may be included in any filter or similar mesh as the wire mesh 72 shown in FIG. 9.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A vascular access device, comprising:
a body,
an interior chamber disposed within the body for receiving a fluid, and
a filter within the interior chamber for filtering a pathogen within the fluid.

2. The vascular access device of claim 1, further comprising:
an antimicrobial agent within the interior chamber,
wherein the filter is impervious to the antimicrobial agent, and
wherein the filter borders the antimicrobial agent on at least a first side.

3. The vascular access device of claim 1, wherein the filter includes an electrical multilayer screen.

4. The vascular access device of claim 1, wherein the filter includes a biocide barb.

5. The vascular access device of claim 4, wherein the filter includes multiple layers of biocide barbs.

6. The vascular access device of claim 1, wherein the filter prevents the passage of any agent the size of a pathogen.

7. The vascular access device of claim 1, wherein the filter is treated with an antimicrobial agent.

8. A method of filtering a pathogen in a vascular access device, comprising:
providing a vascular access device having an interior chamber for receiving a fluid,
providing a filter within the interior chamber of the vascular access device,
moving the fluid through the filter, and
filtering a pathogen as the fluid moves through the filter.

9. The method of claim 8, further comprising:
providing an antimicrobial agent within the interior chamber, and
bordering the antimicrobial agent with the filter on at least a first side of the filter,
wherein the filter is impervious to the antimicrobial agent.

10. The method of claim 8, wherein filtering includes electrocuting the pathogen as it moves through the filter.

11. The method of claim 8, wherein filtering includes cutting the pathogen as it moves through the filter.

12. The method of claim 8, wherein the filter includes multiple layers of biocide barbs.

13. The method of claim 8, wherein filtering includes preventing the passage through the filter of any agent the size of the pathogen.

14. The method of claim 8, wherein the filter is treated with an antimicrobial agent.

15. A medical device, comprising:
means for accessing the vascular system of a patient, and
means for filtering a pathogen, wherein the means for filtering the pathogen is located within the means for accessing the vascular system of the patient.

16. The medical device of claim 15, wherein the means for filtering a pathogen includes a means for killing a pathogen bordered by a means for retaining the means for killing within the means for accessing.

17. The medical device of claim 15, wherein the means for filtering includes means for electrocuting the pathogen.

18. The medical device of claim 15, wherein the means for filtering includes means for cutting the pathogen.

19. The medical device of claim 15, wherein the means for filtering includes means for preventing the passage of any agent the size of the pathogen.

20. The medical device of claim 15, wherein the means for filtering includes a biocidal coating.
